(19) United States
(12) Patent Application Publication
(10) Pub. No.: US 2026/0130608 A1
(43) Pub. Date: May 14, 2026

(54) METHOD OF ADAPTIVE INVERSE TONAL AUDIBILITY LEVEL SCREENING

(71) Applicant: HEARING REHABILITATION CENTER "MELFON", LTD., Moscow (RU)

(72) Inventors: Valeriy Stepanovich SIROTKIN, g Moskva (RU); Vladimir Vladimirovich KHANYKOV, g Moskva (RU); Sergey Mikhailovich PASYUTA, g. Moskva (RU)

(73) Assignee: HEARING REHABILITATION CENTER "MELFON", LTD., Moscow (RU)

(21) Appl. No.: 19/327,111

(22) Filed: Sep. 12, 2025

(30) Foreign Application Priority Data
Nov. 14, 2024 (RU) 2024134030

(51) Int. Cl. A61B 5/12; A61B 5/00

(52) U.S. Cl. CPC A61B 5/123; A61B 5/7475

(57) ABSTRACT

The present invention relates to medicine, namely to surdology-otorhinolaryngology, and can be used in conducting audiometry for diagnostic purposes during mass preventive examinations of the population. The patient is consistently presented with sound signals in the frequency range: 125-250-500-750-1000-2000-3000-4000-6000-8000-10000-12000-16000 Hz at three tone levels of 5, 10, 20, 35 and 50 dB. At the same time, they start with a 20 dB tone signal. at the same time, they start with a 20 dB tone signal—test 0. If, as a result of the “0” test, the patient replied “There is a signal” at all the presented frequencies, then perform the “−1” test with a 10 dB tone level, if the patient replied “There is a signal” at all the presented frequencies, proceed to the “−2” test with a 5 dB tone level, and if the patient replied “There is a signal” at all the presented frequencies. There is a signal” at all the presented frequencies, then the study is completed and “Excellent audibility” is determined. If, as a result of the “−2” test, the patient receives a response of “No signal” at least one of the frequencies, then the study is completed and “good audibility” is determined. If, as a result of the “−1” test, the response “No signal” is received from the patient at least on one of the frequencies, then the study is completed and the “Hearing norm” is determined; if, as a result of the “0” test, if the response “No signal” is received from the patient at least on one of the frequencies, then the “1” test is performed.” with a tone level of 35 dB; if, as a result of the “1” test, the patient replied “There is a signal” at all the presented frequencies, then the study is completed and a potential “mild hearing impairment” is determined. If, as a result of test “1”, the patient replied “There is no signal” at at least one of the frequencies, then test “2” is performed with a tone level of 50 dB. If, as a result of the “2” test, the patient replied “There is a signal” at all the presented frequencies, then the study is completed and the “average hearing impairment” is determined. If, as a result of the “2” test, the patient replied “There is no signal” at least on one of the frequencies, then the study is completed and a “high hearing impairment” is determined. The method allows for an express hearing assessment in a short time period during mass preventive examinations of the population.

### FIELD OF TECHNOLOGY

The present invention relates to medicine, namely to sign language-otorhinolaryngology, and can be used in conducting audiometry for diagnostic purposes during mass preventive examinations of the population.

### BACKGROUND

To date, tonal hearing screening is almost the only way to obtain “at least some” instrumental assessment of auditory perception in systematically conducted mass examinations in schools. Successful mastering of educational material largely determines not only the successful future of a student, but also the level of future scientific and technological development of the society in which today's students will work. Numerous studies have proven that even a slight hearing impairment significantly reduces the ability to assimilate educational material. WHO has developed the criterion of “educationally Significant Hearing Loss”—ESHL “Educationally Significant Hearing Loss” (AAA, 2011; ASHA, 1997).

The ESHL level is considered to be hearing loss, which interferes with student academic performance (WHO, 2014). This may include permanent sensorineural, conductive, and mixed hearing loss, as well as transient conductive loss. However, the severity of hearing loss that ESHL represents is not always clearly defined. According to the World Health Organization (2014), disabling hearing loss in children is the average threshold of hearing in the best ear at frequencies of 0.5, 1, 2, 4 kHz, which is >30 dB HL.”

In order to reduce labor costs, all existing methods use a narrowed range of the studied “speech” frequencies: 500, 1000, 2000, 4000 Hz, although it is already a well-known fact that high speech frequencies over 6000 Hz have a great influence on speech intelligibility.

The use of manually operated audiometers leads to significant time costs, which leads to the need to use only one volume level value of the scanning test signal. The prognostic effectiveness of the method is sharply reduced and the burden on the healthcare system is greatly increased due to the large number of unconfirmed referrals for additional examination.

From the prior art, a method of two-stage examination of hearing organs in preschool children. At the first stage, the tympanometry method is used, at the second stage, the otoacoustic emission method is used at the distortion product frequency. The examination begins with the use of the tympanometry method at a probing tone frequency of 226 Hz with a pressure change rate from +200 to −400 daPa/s. According to the tympanograms obtained, which have a peak dependence of static compliance on changes in positive or negative air pressure in the external auditory canal, the pressure in daPa/c is estimated, at which the peak of compliance is recorded. If the pressure in the tympanic cavity and the external auditory canal equalizes, and the pressure at which the peak of compliance is recorded ranges from +50 to −100 daPa/c, the function of the middle ear is assessed as normal. Otoacoustic emission examination is continued. If a pathology of the middle ear is detected according to the results of tympanometry, the child is referred for treatment of the pathology of the middle ear. Then, after 3 weeks, repeated tympanometry is performed and the examination is continued using the otoacoustic emission method at the frequency of the distortion product. The registration criterion is the ratio of the emission power to the background noise power of +6 dB in 70% of the frequency bands. If pathology is detected based on the results of otoacoustic emission at the frequency of the distortion product, the child is sent to a sign language center for examination. The method makes it possible to increase the reliability and objectivity of monitoring hearing aid pathology in preschool children (RU 2759485 C1 Nov. 15, 2021).

From the prior art, there is a known method and software and hardware complex for performing diagnostic procedures in terms of performing a pre-medical preliminary classifying multifactorial assessment of the capabilities of a human auditory analyzer during mass preventive examinations of the population. A method is proposed for pre-medical preliminary classifying multifactorial assessment of the capabilities of a human auditory analyzer during mass preventive examinations of the population, performed using a computing device connected to audio playback devices and containing stages in which: using a computing device, a primary test speech sequence (TSS) is formed, which consists of sentences consisting of the first number of words, based on the matrix test; generate a competing noise sound for the primary TSS; the primary TSS is reproduced using audio signal reproduction devices made in the form of air and bone sound transmission headphones, while the TSS is reproduced simultaneously with the noise competing sound at the first signal-to-noise ratio using speech simulation based on a deep machine learning model; the user's oral response is received; the user's oral response is automatically analyzed by recognizing the TSS converting it to text format and analyzing the correctness of the answer using a machine learning model; moreover, based on the analysis of the user's oral responses, a dynamic change in the complexity of the assessment is carried out, in which, as a result of each automatic analysis, the number of words in sentences forming the TSS and/or the signal-to-noise ratio of the reproduced signal is changed; the user's auditory analyzer is evaluated based on the responses during playback of the test speech sequence. The invention provides an automated pre-medical preliminary classifying assessment of the possibility of a human auditory analyzer during mass preventive examinations of the population (RU 2765108 C1 Jan. 25, 2022).

In addition, a method is known from the prior art in which screening audiometry is performed by applying in semi-automatic mode a sequence of tone signals of a standard set of frequencies separately into each of the channels of the playback device with recording of the user's response by recognizing tone signals.

Reproduction of a test sequence of monophonic signals in an extended frequency range for the construction of a screening audiogram (RU 2743049 C1 Feb. 15, 2021).

The disadvantages of this development is a single test sequence at a certain preset test signal level. All frequencies declared for the survey are scanned sequentially. This is a traditional method of tonal audiometry screening, performed on an audiometer with manual adjustment of the volume level of the test signal. During the examination of a group of patients, the required level of the test signal is set and the entire group is examined at this value. If it is necessary to change the level, the procedure is repeated. At each pass, the patient is tested at only one level of the test signal.

Based on the consideration of the identified problematic issues of existing methodological solutions for the organization of screening examinations of schoolchildren (minors), it is possible to formulate requirements for a promising option for tonal screening of auditory perception:
1. It is necessary to abandon the use of a fixed grid of 4 frequencies. For each patient, it is necessary to determine the upper limit of the perceived tonal signals. It is necessary to identify and correct not only “bad” hearing, but also to take care of identifying and maintaining “excellent” hearing.
2. Multi-level organization of screening. The number of hearing scanning levels performed per headphone installation must be adjusted depending on the objectives of the examination. For example, for conducting examinations in schools, we can recommend a three-level screening with sequential testing of one patient at three levels of test signal intensity, providing an operational possibility of automatic “traffic light” classification of the level of his perception of tonal signals:
   “Green level”—no signs of abnormalities were detected.;
   “Yellow level”—signs of a slight impairment in the perception of speech range tonal signals were detected;
   “Red level”—signs of impaired perception of tonal signals have been identified. Repeated in-depth examination is necessary.
3. The maximum time of the actual testing process for one patient (from the moment the “start” button is pressed to the end of the test) should not exceed 1.5 minutes for a three-level scan of a mass school student (assuming no neuropsychiatric behavioral abnormalities).

### SUMMARY

The claimed invention solves a technical problem in terms of ensuring the detection of persons with normal hearing, but with a tendency to decrease, especially in the field of high speech frequencies, as well as to identify more severe hearing disorders, in addition, the objective of this solution is to reduce the study time. Thus, the claimed method allows for early diagnosis of hearing loss when a drop in high frequencies is noticed compared to previous measurements of longitudinal analysis.

Technically, the result is to enable rapid hearing assessment in a short period of time during mass preventive examinations of the population, improve diagnostic accuracy through an expanded frequency range, and perform early diagnosis of hearing loss when a drop in high frequencies is detected compared to previous measurements longitudinal analysis. The reduction in examination time is ensured by the fact that the patient is presented with an extended frequency range of three tones per study, so instead of examining all patients one at a time at the same testing level, followed by resetting the level (again the same for all), with headphones reinstalling (plus time for ongoing disinfection), it is proposed to perform the entire range of testing for all the frequencies and levels of the test signal of interest for each patient.

The claimed method, due to the bidirectional test and adaptability, makes it possible to obtain a documented hearing assessment for each patient in one pass.

The claimed technical result is achieved through the following techniques.

The study begins with a common “0” test for all patients. The level and frequency range of the next test depends on the result of the previous scan run. It can change:
in the direction of reducing the hearing thresholds, if the patient answered “There is a signal” on the first pass at all frequencies. The goal is to determine how well a person hears faint sounds in an extended frequency range: 125 Hz-16 kHz.
in the direction of increasing the hearing thresholds, if on the first pass, at least one frequency will have the answer “No signal”. The goal is the inverse of the previous option—the worst case scenario is determined.

A method of adaptive inverse tonal screening of the hearing level, including the presentation of audio signals to the patient in automatic mode in the frequency range when the sound signal levels change, characterized in that the range of tested frequencies is used: 125 Hz, 250 Hz, 500 Hz, 750 Hz, 1000 Hz, 2000 Hz, 3000 Hz, 4000 Hz, 6000 Hz 8000 Hz, 10000 Hz, 12000 Hz, 16000 Hz and sequentially through headphones, the patient is presented with a signal at tone levels of 5, 10, 20, 35 and 50 dB, while starting with a tone signal of 20 dB—test 0,
the “0” test is performed at 500 Hz, 1000 Hz, 2000 Hz, 4000 Hz, 6000 Hz, 8000 Hz, and if, as a result of the “0” test, the patient replied “There is a signal” at all the presented frequencies, then the “−1” test is performed with a tone level of 10 dB and at frequencies of 125 Hz, 250 Hz, 500 Hz, 750 Hz, 1000 Hz, 2000 Hz, 3000 Hz, 4000 Hz, 6000 Hz, 8000 Hz, 10000 Hz, 12000 Hz, 16000 Hz,
if the patient replied “There is a signal” at all the presented frequencies of the “−1” test, then the “−2” test is performed—with a tone level of 5 dB and at frequencies of 125 Hz, 250 Hz, 500 Hz, 750 Hz, 1000 Hz, 2000 Hz, 3000 Hz, 4000 Hz, 6000 Hz, 8000 Hz, 10000 Hz, 12000 Hz, 16000 Hz,
if the patient answered “There is a signal” during the −2 test at all the presented frequencies, then the study is completed and “Excellent audibility” is determined;
moreover, if, as a result of the “−2” test, the response “No signal” is received from the patient at least on one of the frequencies, then the study is completed and “good audibility” is determined;
if, as a result of the “−1” test, a “No signal” response is received from the patient at least on one of the frequencies, then the study is completed and the “Hearing norm” is determined;
at the same time, if, as a result of the “0” test, a “No signal” response is received from the patient on at least one of the frequencies, then test “1” with a tone level of 35 dB at frequencies 125 Hz, 250 Hz, 500 Hz, 1000 Hz, 2000 Hz, 4000 Hz, 6000 Hz, during which if the patient replied “There is a signal” at all the presented frequencies, the study is completed and a potential “mild hearing impairment” is determined;
if, as a result of test “1”, the patient replied “No signal” at at least one of the frequencies, then test “2” is performed with a tone level of 50 dB and at frequencies of 125 Hz, 250 Hz, 500 Hz, 1000 Hz, 2000 Hz, 4000 Hz, 6000 Hz, during which if the patient replied “There is a signal” at all the presented frequencies, then the study is completed and the “average hearing impairment” is determined.

At the same time, if, as a result of test “2”, the patient replied “There is no signal” at least one of the frequencies, then the study is completed and the “high hearing impairment” is determined.

In this case, air or bone sound transmission headphones can be used.

### A BRIEF DESCRIPTION OF THE DRAWINGS

FIG. 1 is an adaptive inverse tonal screening of the hearing level of patient N from Example 1.

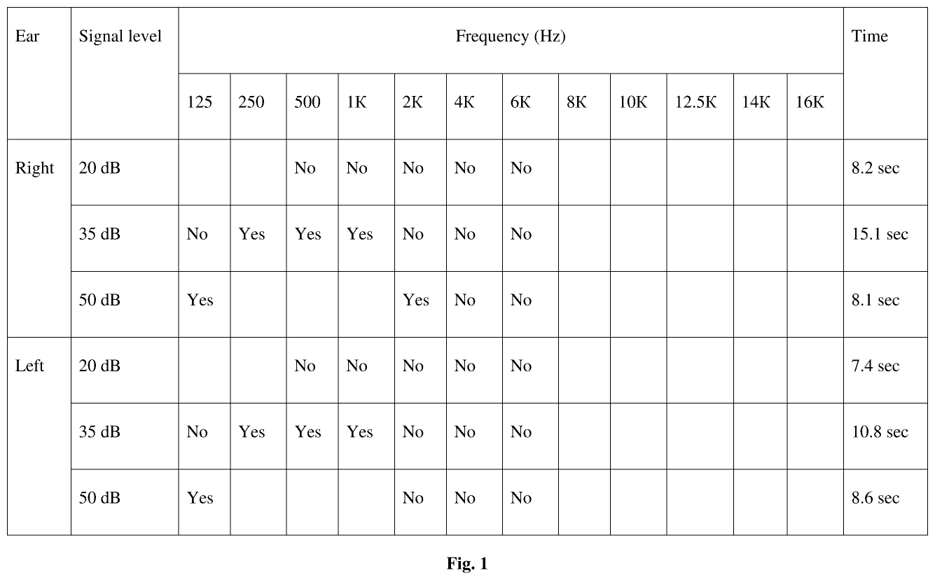

FIG. 2 shows adaptive inverse tonal screening of patient A's hearing level from Example 2.

FIG. 3 shows adaptive inverse tonal screening of the hearing level in patient G From Example 3

FIG. 4 shows adaptive inverse tonal screening of the hearing level of patient P from Example 4.

FIG. 5 shows adaptive inverse tonal screening of the hearing level in patient A from Example 5.

FIG. 6 shows adaptive inverse tonal screening of the hearing level in patient M from Example 6.

### DETAILED DESCRIPTION OF THE INVENTION

To eliminate the existing shortcomings in conducting rapid hearing assessment, in order to organize multi-level screening and examination, for example, in schools, we can recommend three-level screening with sequential testing of one patient at three levels of test signal intensity, providing an operational possibility of automatic classification of the level of his perception of tonal signals. This screening allows you to significantly reduce the time of the actual testing process for one patient with a three-level scan of a mass school student. In addition, the method makes it possible to carry out an early diagnosis of hearing loss when a drop in high frequencies is noticed compared to previous measurements of longitudinal analysis.

The study begins with a common “0” test for all patients. The level and frequency range of the next test depends on the result of the previous scan run. It can change:
in the direction of decreasing the hearing thresholds, if on the first pass at all frequencies the patient replied “There is a signal”. The goal is to determine how well a person hears faint sounds in an extended frequency range: 125 Hz-16 kHz.
in the direction of increasing the hearing thresholds, if on the first pass, at least one frequency will have the answer “No signal”. The goal is the inverse of the previous option—the worst case scenario is determined.

Any response of “No signal” is confirmed. The control signal of the same level is repeated at the same frequency. In the case of a “discrepancy” between the main and control signals, another control test is performed and the result is evaluated by two identical answers out of 3.

TABLE 1
Frequency ranges and recommended (default) scan levels

| Test | Signal level | | |
|---|---|---|---|
| No | (dB) | Frequency range | Estimate |
| −2 | 5 | 125 Hz, 250 Hz, 500 Hz, 750 Hz, 1000 | There is a signal on all |
| | | Hz, 2000 Hz, 3000 Hz, 4000 Hz, 6000 | frequencies - excellent |
| | | Hz, 8000 Hz, 10000 Hz, 12000 Hz, | audibility |
| | | 16000 Hz | There is no signal, at least on |
| | | | one of the frequencies - good |
| | | | audibility |
| −1 | 10 | 125 Hz, 250 Hz, 500 Hz, 750 Hz, 1000 | There is no signal, at least on |
| | | Hz, 2000 Hz, 3000 Hz, 4000 Hz, 6000 | one of the frequencies - normal |
| | | Hz, 8000 Hz, 10000 Hz, 12000 Hz, | audibility |
| | | 16000 Hz | |
| 0 | 20 | 500 Hz, 1000 Hz, 2000 Hz, 4000 Hz, | |
| | | 6000 Hz, 8000 Hz | |
| 1 | 35 | 125 Hz, 250 Hz, 500 Hz, 1000 Hz, | There is a signal on all the |
| | | 2000 Hz, 4000 Hz, 6000 Hz | presented frequencies - a slight |
| | | | hearing impairment. |
| 2 | 50 | 125 Hz, 250 Hz, 500 Hz, 1000 Hz, | There is a signal on all the |
| | | 2000 Hz, 4000 Hz, 6000 Hz | presented frequencies for |
| | | | hearing impairment |
| | | | There is no signal, at least on |
| | | | one of the presented frequencies - |
| | | | high hearing impairment |

The patient automatically receives audio signals in the frequency range when the audio signal levels change, characterized in that the range of tested frequencies is used: 125 Hz, 250 Hz, 500 Hz, 750 Hz, 1000 Hz, 2000 Hz, 3000 Hz, 4000 Hz, 6000 Hz, 8000 Hz, 10000 Hz, 12000 Hz, 16000 Hz and sequentially through headphones, the patient is presented with a signal at tone levels of 5, 10, 20, 35 and 50 dB, while starting with a tone signal of 20 dB—test 0,
perform the “0” test at frequencies of 500 Hz, 1000 Hz, 2000 Hz, 4000 Hz, 6000 Hz, 8000 Hz, and if, as a result of the “0” test, the patient replied “There is a signal” at all presented frequencies, then perform the “−1” test with a tone level of 10 dB and at frequencies of 125 Hz, 250 Hz, 500 Hz, 750 Hz, 1000 Hz, 2000 Hz, 3000 Hz, 4000 Hz, 6000 Hz, 8000 Hz, 10000 Hz, 12000 Hz, 16000 Hz,
if the patient replied “There is a signal” at all the presented frequencies of the “−1” test, then the “−2” test is performed—with a tone level of 5 dB and at frequencies of 125 Hz, 250 Hz, 500 Hz, 750 Hz, 1000 Hz, 2000 Hz, 3000 Hz, 4000 Hz, 6000 Hz, 8000 Hz. Hz, 10000 Hz, 12000 Hz, 16000 Hz,
if the patient during the “−2” test replied “There is a signal” at all the presented frequencies, then the study is completed and “Excellent audibility” is determined;
moreover, if, as a result of the “−2” test, the response “No signal” is received from the patient at least on one of the frequencies, then the study is completed and “good audibility” is determined;
if, as a result of the “−1” test, a “No signal” response is received from the patient at least on one of the frequencies, then the study is completed and the “Hearing norm” is determined;
at the same time,
if, as a result of the “0” test, a “No signal” response is received from the patient on at least one of the frequencies, then test “1” with a tone level of 35 dB at frequencies 125 Hz, 250 Hz, 500 Hz, 1000 Hz, 2000 Hz, 4000 Hz, 6000 Hz, during which if the patient replied “There is a signal” at all the presented frequencies, the study is completed and a potential “mild hearing impairment” is determined;
if, as a result of test “1”, the patient replied “No signal” at at least one of the frequencies, then test “2” is performed with a tone level of 50 dB and at frequencies of 125 Hz, 250 Hz, 500 Hz, 1000 Hz, 2000 Hz, 4000 Hz, 6000 Hz, during which if the patient replied “There is a signal” at all the presented frequencies, then the study is completed and the “average hearing impairment” is determined;
at the same time, if, as a result of the “2” test, the patient replied “There is no signal” at least on one of the frequencies, then the study is completed and a “high hearing impairment” is determined.

In a particular case, headphones are air or bone sound transmission.

Thus, the bidirectional test and adaptability make it possible to obtain a documented hearing assessment for each patient in one pass.

The claimed method can be implemented based on the MELFON hardware and software package (RU 2743049 C1 Feb. 15, 2020).

Along with the above, the claimed method allows, instead of examining all patients one by one at the same test level, followed by resetting the level (again the same for all), with the headphones reinstalled (plus time for ongoing disinfection), it is proposed to perform the entire test package at all frequencies and test signal levels of interest for each patient.

In addition, not only patients with auditory perception disorders are identified, but also patients with a normal level of perception of tonal signals in the extended range up to 16 kHz are identified. It is important not only to treat or correct hearing disorders, but also to ensure the preservation of hearing.

The upper limit of sound perception thresholds is determined for each patient. This indicator is critically important in developing a correction scheme for the detected degree of hearing loss.

### Example 1

Patient N. underwent adaptive inverse tonal screening of hearing level. During which audio signals are automatically presented to the patient via headphones at each frequency when the audio signal levels change, using the following frequencies: 125 Hz, 250 Hz, 500 Hz, 750 Hz, 1000 Hz, 2000 Hz, 3000 Hz, 4000 Hz, 6000 Hz, 8000 Hz, 10000 Hz, 12000 Hz, 16000 Hz. They start with the “0” test with a 20 dB tone level at frequencies of 500 Hz, 1000 Hz, 2000 Hz, 4000 Hz, 6000 Hz, 8000 Hz. As a result of the test, the patient stated that he does not hear a signal on any of the frequencies with both his right and left ears. In this case, we switched to test “1” with a tone level of 35 dB at frequencies 125 Hz, 250 Hz, 500 Hz, 1000 Hz, 2000 Hz, 4000 Hz, 6000 Hz, during which, if the patient replied “There is a signal” at all presented frequencies, 250 Hz, 500 Hz, 1000 Hz With both the right and left ears, the patient replied at the remaining frequencies that “there is no signal.” They proceed to test “2” with a tone level of 50 dB and at frequencies of 125 Hz, 250 Hz, 500 Hz, 1000 Hz, 2000 Hz, 4000 Hz, 6000 Hz, during which, if the patient replied “There is a signal” at frequencies of 125 Hz and 2000 Hz at frequencies of 4000 Hz and 6000 Hz-“there is no signal”, with the right ear, and with the left: There is a signal” at frequencies of 125 Hz, and at frequencies of 2000 Hz, 4000 Hz and 6000 Hz-“there is no signal”. As a result of the test, patient N. has a “high hearing impairment.

### Example 2

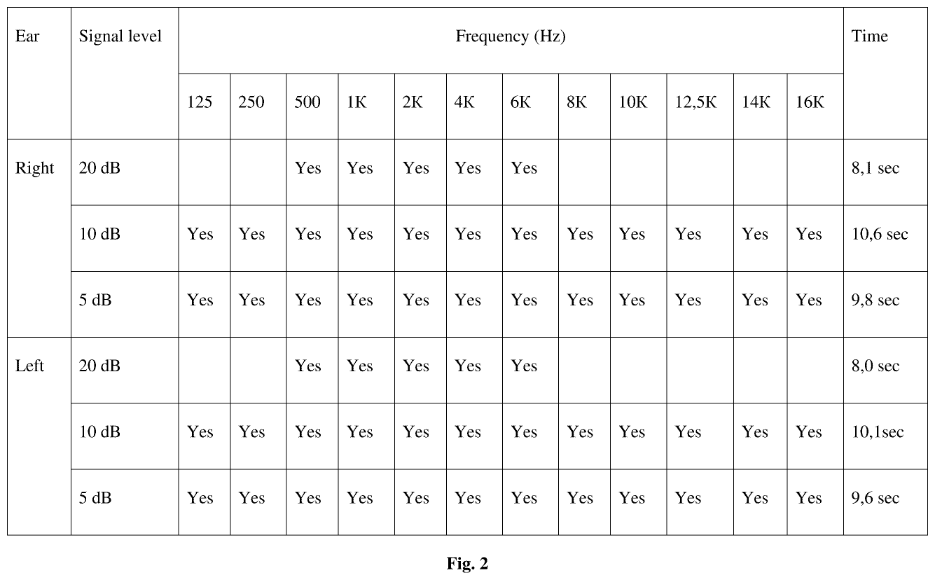

Patient A. underwent adaptive inverse tonal audibility screening using the claimed method, which revealed excellent audibility in both ears, the results are shown in FIG. 2.

### Example 3

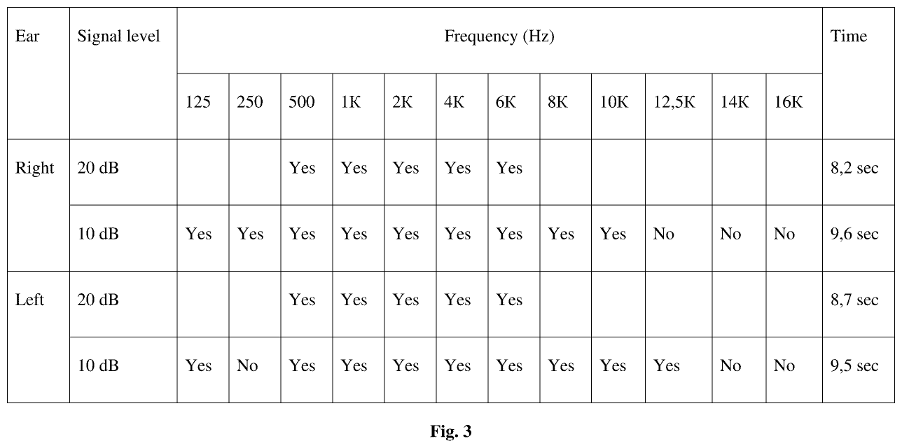

Patient G. underwent adaptive inverse tonal audibility screening using the claimed method, which revealed normal audibility in both ears, the results are shown in FIG. 3.

### Example 4

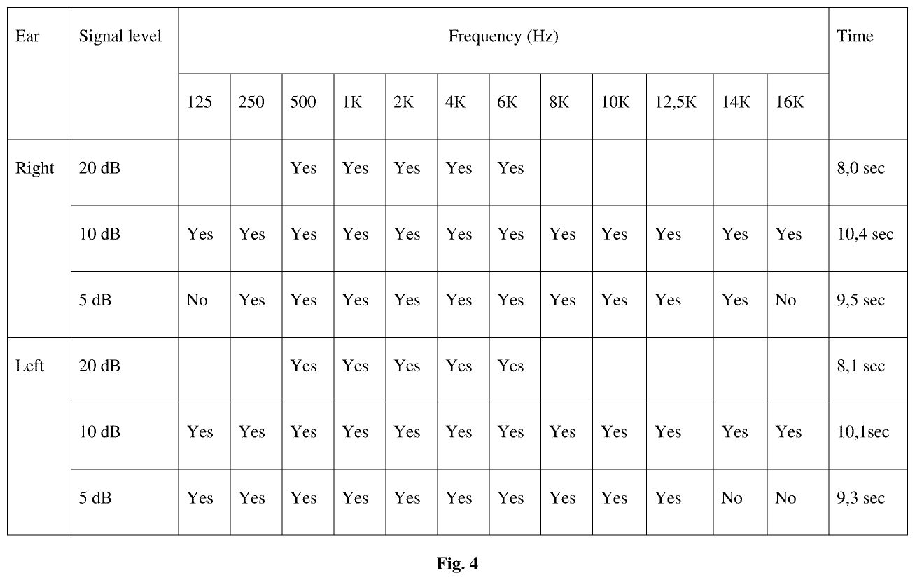

Patient P. underwent adaptive inverse tonal screening of the level of audibility using the claimed method, during which good audibility was detected in both ears, the results are shown in FIG. 4.

### Example 5

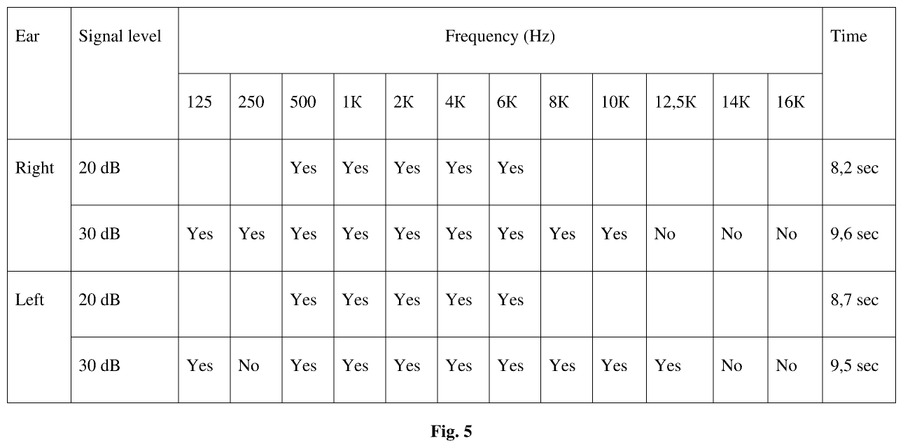

Patient A. underwent adaptive inverse tonal screening of the audibility level using the claimed method, during which a slight hearing impairment was detected in both ears, the results are shown in FIG. 5.

### Example 6

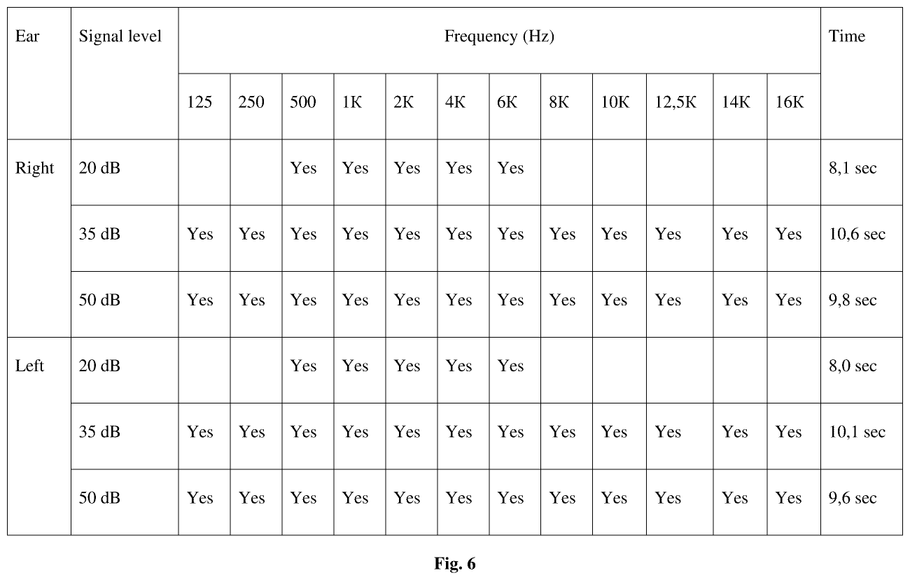

Patient M. underwent adaptive inverse tonal screening of the audibility level using the claimed method, during which an average hearing impairment was detected in both ears, the results are shown in FIG. 6.

1. A method of adaptive inverse tonal screening of the audibility level, including the presentation of audio signals to the patient in automatic mode at each frequency when the audio signal levels change, using frequencies: 125 Hz, 250 Hz, 500 Hz, 750 Hz, 1000 Hz, 2000 Hz, 3000 Hz, 4000 Hz, 6000 Hz, 8000 Hz Hz, 10000 Hz, 12000 Hz, 16000 Hz, to generate test sequences of signals presented via headphones to the patient, with tone levels of 5, 10, 20, 35 and 50 dB, depending on the test being performed, while
perform the “0” test with a 20 dB tone level at 500 Hz, 1000 Hz, 2000 Hz, 4000 Hz, 6000 Hz, 8000 Hz, and if, as a result of the “0” test, the patient replied “There is a signal” at all presented frequencies, then perform the “−1” test with a tone level 10 dB and at frequencies of 125 Hz, 250 Hz, 500 Hz, 750 Hz, 1000 Hz, 2000 Hz, 3000 Hz, 4000 Hz, 6000 Hz, 8000 Hz, 10000 Hz, 12000 Hz, 16000 Hz,
if the patient replied “There is a signal” at all the presented frequencies of the “−1” test, then the “−2” test is performed—with a tone level of 5 dB and at frequencies of 125 Hz, 250 Hz, 500 Hz, 750 Hz, 1000 Hz, 2000 Hz, 3000 Hz, 4000 Hz, 6000 Hz, 8000 Hz. Hz, 10000 Hz, 12000 Hz, 16000 Hz,
if the patient during the “−2” test replied “There is a signal” at all the presented frequencies, then the study is completed and “Excellent audibility” is determined;
moreover, if, as a result of the “−2” test, the response “No signal” is received from the patient at least on one of the frequencies, then the study is completed and “good audibility” is determined;
if, as a result of the “−1” test, a “No signal” response is received from the patient at least on one of the frequencies, then the study is completed and the “Hearing norm” is determined;
at the same time,
if, as a result of the “0” test, a “No signal” response is received from the patient on at least one of the frequencies, then test “1” with a tone level of 35 dB at frequencies 125 Hz, 250 Hz, 500 Hz, 1000 Hz, 2000 Hz, 4000 Hz, 6000 Hz, during which if the patient replied “There is a signal” at all the presented frequencies, the study is completed and a potential “mild hearing impairment” is determined;
if, as a result of test “1”, the patient replied “No signal” at at least one of the frequencies, then test “2” is performed with a tone level of 50 dB and at frequencies of 125 Hz, 250 Hz, 500 Hz, 1000 Hz, 2000 Hz, 4000 Hz, 6000 Hz, during which if the patient replied “There is a signal” at all the presented frequencies, then the study is completed and the “average hearing impairment” is determined;
at the same time, if, as a result of the “2” test, the patient replied “There is no signal” at least on one of the frequencies, then the study is completed and a “high hearing impairment” is determined.

2. The method according to claim 1, wherein the headphones are air or bone sound transmission.
